(19) United States
(12) Patent Application Publication
(10) Pub. No.: US 2009/0030325 A1
(43) Pub. Date: Jan. 29, 2009

(54) ULTRASONIC PROBE, ULTRASONIC ENDSCOPE, AND ULTRASONIC DIAGNOSTIC APPARATUS

(75) Inventor: Hiroaki HYUGA, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 11/865,251

(22) Filed: Oct. 1, 2007

(30) Foreign Application Priority Data
Sep. 29, 2006 (JP) 2006-267330

(51) Int. Cl. A61B 8/14

(52) U.S. Cl. 600/459

(57) ABSTRACT

In an ultrasonic probe to be used in an ultrasonic diagnostic apparatus for medical use, ultrasonic transducers are cooled while sufficiently absorbing ultrasonic waves released to the back of the ultrasonic transducers without causing attenuation of ultrasonic waves transmitted or received by the ultrasonic transducers. The ultrasonic probe includes: an ultrasonic transducer array including plural ultrasonic transducers for transmitting and receiving ultrasonic waves; an acoustic matching layer provided on a front of the ultrasonic transducer array; a cooling mechanism directly or indirectly provided on a back of the ultrasonic transducer array and including a porous member; a backing material provided on the back of the ultrasonic transducer array via at least the cooling mechanism; and channels for circulation of a liquid heat transfer material in the cooling mechanism.

### BACKGROUND OF THE INVENTION

1. Field of the Invention

The present invention relates to an ultrasonic probe to be used when intracavitary scanning or extracavitary scanning is performed on an object to be inspected, and an ultrasonic endoscope to be inserted into a body cavity of the object. Further, the present invention relates to an ultrasonic diagnostic apparatus including such an ultrasonic probe or ultrasonic endoscope and an ultrasonic diagnostic apparatus main body.

2. Description of a Related Art

In medical fields, various imaging technologies have been developed in order to observe the interior of an object to be inspected for making diagnoses. Especially, ultrasonic imaging for acquiring interior information of the object by transmitting and receiving ultrasonic waves enables image observation in real time and provides no exposure to radiation unlike other medical image technologies such as X-ray photography or RI (radio isotope) scintillation camera. Accordingly, ultrasonic imaging is utilized as an imaging technology at a high level of safety in a wide range of departments including not only the fetal diagnosis in the obstetrics, but gynecology, circulatory system, digestive system, etc.

The ultrasonic imaging is an image generation technology utilizing the nature of ultrasonic waves that the ultrasonic waves are reflected at a boundary between regions with different acoustic impedances (e.g., a boundary between structures). Typically, an ultrasonic diagnostic apparatus (or referred to as an ultrasonic imaging apparatus or an ultrasonic observation apparatus) is provided with an ultrasonic probe to be used in contact with the object or ultrasonic probe to be inserted into a body cavity of the object. Alternatively, the apparatus may be provided with an ultrasonic endoscope in combination of an endoscope for optically observing the interior of the object and an ultrasonic probe for intracavitary.

Using such an ultrasonic probe or ultrasonic endoscope, ultrasonic beams are transmitted toward the object such as a human body and ultrasonic echoes generated by the object are received, and thereby, ultrasonic image information is acquired. On the basis of the ultrasonic image information, ultrasonic images of structures (e.g., internal organs, diseased tissues, or the like) existing within the object are displayed on a display unit of the ultrasonic diagnostic apparatus.

In the ultrasonic probe, a vibrator (piezoelectric vibrator) having electrodes formed on both sides of a material (a piezoelectric material) that expresses piezoelectric effect is generally used as an ultrasonic transducer for transmitting and receiving ultrasonic waves. As the piezoelectric material, a piezoelectric ceramics represented by PZT (Pb(lead)zirconate titanate), a polymeric piezoelectric material represented by PVDF (polyvinylidene difluoride), or the like is used.

When a voltage is applied to the electrodes of the vibrator, the piezoelectric material expands and contracts due to the piezoelectric effect to generate ultrasonic waves. Accordingly, plural vibrators are one-dimensionally or two-dimensionally arranged and the vibrators are sequentially driven, and thereby, an ultrasonic beam transmitted in a desired direction can be formed. Further, the vibrator receives the propagating ultrasonic waves, expands and contracts to generate an electric signal. The electric signal is used as a reception signal of ultrasonic waves.

When ultrasonic waves are transmitted, drive signals having great energy are supplied to the ultrasonic transducers. Not the whole energy of the drive signals is converted into acoustic energy and the considerable amount of energy turns into heat. Thus, there has been a problem of rising temperature of the ultrasonic probe during its use. However, the ultrasonic probe for medical use is used in direct contact with a living body of human or the like, and the surface temperature of the ultrasonic probe is requested to be 43° C. or below for safety reasons of prevention of low-temperature burn.

As a related technology, Japanese Patent Application Publication JP-P2002-291737A discloses an ultrasonic probe having an ultrasonic probe head part for transmitting and receiving ultrasonic waves, a cable electrically connected to the ultrasonic probe head part, and a cable cooling part thermally connected to at least part of the cable.

However, in JP-P2002-291737A, only a small portion of the ultrasonic probe head part is indirectly cooled via the cable by the cable cooling, and therefore, the cooling efficiency is not very good.

Japanese Patent Application Publication JP-A-63-242246 discloses an ultrasonic probe for intracavitary to be inserted into a body cavity for imaging ultrasonic images, and the ultrasonic probe is provided with cooling means for cooling the heat generated by an ultrasonic converter during operation of the ultrasonic probe, in a predetermined position of a sound absorbing material. In JP-A-63-242246, a cooling pipe is provided in the ultrasonic probe and a cooling medium such as water is flown through the pipe, and thereby, the group of ultrasonic vibrators are cooled.

However, when the cooling pipe is provided on the side of the group of ultrasonic vibrators (FIG. 3), the thermal coupling between the cooling pipe and the group of ultrasonic vibrators becomes weaker and the cooling efficiency is not good. On the other hand, when the cooling pipe is provided on the back of the group of ultrasonic vibrators (FIGS. 4-6), there is a fear that the ultrasonic waves released to the back of the group of ultrasonic vibrators may not be sufficiently absorbed.

Japanese Patent Application Publication JP-A-11-299775 discloses an ultrasonic diagnostic apparatus including transferring means for guiding heat generated in a sound absorbing member to a position apart from the sound absorbing member, and releasing means provided at the position apart from the sound absorbing member, for releasing the heat guided by the transferring means. In the sound absorbing member, a surface opposite to a surface on which ultrasonic vibrators have been provided is formed in a curved configuration having a focus for reflecting and concentrating ultrasonic waves radiated from the ultrasonic vibrators toward the sound absorbing member, and a heat absorbing part of the transferring means is provided in the focus position within the sound absorbing member (FIG. 6).

In JP-A-11-299775, the temperature of the vibrator part at the leading end of an insertion part is controlled by electronic cooling means provided within the grip part of an ultrasonic probe via a heat pump (FIG. 5). Therefore, the vibrator part is indirectly cooled via the heat pump and so on, and therefore, the cooling efficiency is not good.

Japanese Patent Application Publication JP-A-61-58643 discloses an ultrasonic probe having ultrasonic vibrators and a case accommodating the vibrators, and the ultrasonic probe has means for guiding a cooling material to the object contact side of the ultrasonic vibrators.

However, when a cooling medium is flown along a front face of an acoustic lens, that is, through partition walls between the object contact side and the acoustic lens of the ultrasonic probe (FIG. 1), the distance between the ultrasonic vibrators and the object becomes longer and causes attenuation of ultrasonic waves transmitted and received by the ultrasonic vibrators. On the other hand, when a channel for the cooling medium is provided within a back acoustic absorbing material (FIG. 3), there is a fear that the ultrasonic waves released to the back of ultrasonic vibrators may not be sufficiently absorbed. Further, when a channel for the cooling medium is provided between the back acoustic absorbing material and the case (FIG. 5), the thermal coupling between the ultrasonic vibrators and the cooling medium becomes weaker, and therefore, the cooling efficiency is not good.

Japanese Utility Model Application Publication JP-U-57-88073 discloses an ultrasonic probe provided with a path for a cooling medium in contact with the object outside of ultrasonic vibrators.

However, as shown in FIG. 1 of JP-U-57-88073, the path for the cooling medium is provided apart from the space where the ultrasonic vibrators are provided, and therefore, only the periphery of the ultrasonic vibrators is cooled on the object contact surface, and the fact that the object is directly affected by the heat generation of the ultrasonic vibrators is unchanged.

Further, Japanese Utility Model Application Publication JP-U-57-88074 discloses an ultrasonic probe provided, outside of ultrasonic vibrators, with a thermoelectric cooling element in contact with the object, and the thermoelectric cooling element is temperature-controllable for heating or cooling the object by changing the direction of a current flow.

However, as shown in FIG. 1 of JP-U-57-88074, the cooling medium is provided apart from the space where the ultrasonic vibrators are provided, and therefore, only the periphery of the ultrasonic vibrators is cooled on the object contact surface, and the fact that the object is directly affected by the heat generation of the ultrasonic vibrators is unchanged.

Japanese Patent Application Publication JP-P2003-38485A discloses an ultrasonic diagnostic apparatus including an ultrasonic probe provided with ultrasonic vibrators for transmitting and receiving ultrasonic waves, and a channel, through which a medium for transferring heat from the ultrasonic vibrators flows, is formed in the ultrasonic probe and a circulation mechanism for circulating the medium is connected to the channel.

However, in JP-P2003-38485A, a water bag to be filled with water as the cooling medium is disposed at the living body side of the probe (i.e., before the ultrasonic vibrators), and thereby, the distance between the ultrasonic vibrators and the object becomes longer and causes the attenuation of ultrasonic waves to be transmitted and received by the ultrasonic vibrators.

### SUMMARY OF THE INVENTION

Accordingly, in view of the above-mentioned problems, a purpose of the present invention is, in an ultrasonic probe or an ultrasonic endoscope to be used in an ultrasonic diagnostic apparatus for medical use, to cool ultrasonic transducers while sufficiently absorbing ultrasonic waves released to the back of the ultrasonic transducers without causing attenuation of ultrasonic waves transmitted or received by the ultrasonic transducers.

In order to accomplish the purpose, an ultrasonic probe according to one aspect of the present invention includes: an ultrasonic transducer array including plural ultrasonic transducers for transmitting and receiving ultrasonic waves; an acoustic matching layer provided on a front of the ultrasonic transducer array; a cooling mechanism directly or indirectly provided on a back of the ultrasonic transducer array and including a porous member; a backing material provided on the back of the ultrasonic transducer array via at least the cooling mechanism; and channels for circulation of a liquid heat transfer material in the cooling mechanism.

Further, an ultrasonic endoscope according to one aspect of the present invention includes: an ultrasonic transducer array provided in an insertion part formed of a material having flexibility to be inserted into a body cavity of an object to be inspected and including plural ultrasonic transducers for transmitting and receiving ultrasonic waves; an acoustic matching layer provided on a front of the ultrasonic transducer array; a cooling mechanism directly or indirectly provided on a back of the ultrasonic transducer array and including a porous member; a backing material provided on the back of the ultrasonic transducer array via at least the cooling mechanism; and channels for circulation of a liquid heat transfer material in the cooling mechanism.

Furthermore, an ultrasonic diagnostic apparatus according to one aspect of the present invention includes: the above-mentioned ultrasonic probe or ultrasonic endoscope; drive signal supply means for supplying drive signals to the plural ultrasonic transducers, respectively; signal processing means for generating image data representing an ultrasonic image by processing reception signals outputted from the plural ultrasonic transducers, respectively; and heat transfer material circulating means connected to the channels of the ultrasonic probe or ultrasonic endoscope, for collecting the heat transfer material from the ultrasonic probe or ultrasonic endoscope, cooling the collected heat transfer material, and supplying the cooled heat transfer material to the ultrasonic probe or ultrasonic endoscope.

According to the present invention, since the cooling mechanism including the porous member is provided between the ultrasonic transducer array and the backing material, and thereby, the ultrasonic transducers can be cooled while providing matching of acoustic impedances. Therefore, ultrasonic waves released to the back of the ultrasonic transducers can be sufficiently absorbed without causing attenuation of ultrasonic waves transmitted or received by the ultrasonic transducers.

### BRIEF DESCRIPTION OF THE DRAWINGS

FIG. 1 is a perspective view showing an exterior appearance and part of an interior of an ultrasonic probe according to the first embodiment of the present invention;

FIG. 2 shows a configuration of an ultrasonic diagnostic apparatus main body to which the ultrasonic probe according to any one of the first to third embodiments of the present invention is connected;

FIG. 3 shows the interior of the ultrasonic probe according to the first embodiment of the present invention;

FIG. 4 is a partially sectional perspective view showing a single-layer ultrasonic transducer;

FIG. 5 shows an interior of a head part of an ultrasonic probe according to the second embodiment of the e present invention;

FIG. 6 is a partially sectional perspective view showing a multilayered ultrasonic transducer;

FIG. 7 is a diagram for explanation of a modified example of the ultrasonic diagnostic apparatus main body to which the ultrasonic probe according to any one of the first to third embodiments of the present invention is connected;

FIG. 8 is a plan view showing an interior of an ultrasonic probe according to the fourth embodiment of the present invention;

FIG. 9 shows a configuration of an ultrasonic diagnostic apparatus main body to which the ultrasonic probe shown in FIG. 8 is connected;

FIG. 10 is a schematic diagram showing a configuration of an ultrasonic endoscope according to one embodiment of the present invention; and

FIG. 11 is an enlarged view showing the leading end of an insertion part shown in FIG. 10.

### DESCRIPTION OF THE PREFERRED EMBODIMENTS

Hereinafter, embodiments of the present invention will be explained in detail with reference to the drawings. The same reference numbers will be assigned to the same component elements and the description thereof will be omitted.

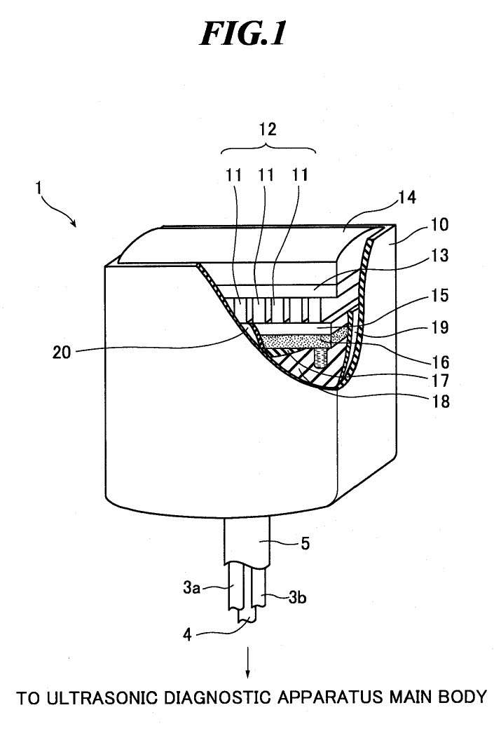

FIG. 1 is a perspective view showing an exterior appearance and part of an interior of an ultrasonic probe according to the first embodiment of the present invention. The ultrasonic probe 1 is used in contact with an object to be inspected when extracavitary scanning is performed. As shown in FIG. 1, a head part of the ultrasonic probe 1 includes a casing 10, an ultrasonic transducer array 12 including plural ultrasonic transducers (vibrators) 11, a first acoustic matching layer 13, an acoustic lens 14, a second acoustic matching layer 15, a micro-channel 16 as a cooling mechanism for cooling the plural ultrasonic transducers 11, a third acoustic matching layer 17, a backing material 18, flexible printed circuits (FPCs) 19 connected to a common electrode of the plural ultrasonic transducers 11, and FPCs 20 connected to signal electrodes of the plural ultrasonic transducers 11.

In the embodiment, in order to cool the plural ultrasonic transducers 11, the micro-channel 16 is formed on the back of the ultrasonic transducer array 12 between the second acoustic matching layer 15 and the third acoustic matching layer 17, and a liquid heat transfer material (heat transfer medium) flowing through the micro-channel 16 cools the ultrasonic transducer array 12. Here, the second acoustic matching layer 15 and the third acoustic matching layer 17 are provided for matching of acoustic impedances in a transfer path of ultrasonic waves from the ultrasonic transducer array 12 via the micro-channel 16 to the backing material 18. Thereby, the ultrasonic waves released to the back of the ultrasonic transducers 11 can be sufficiently absorbed by the backing material 18.

Specifically, given that the acoustic impedance of the vibrators is Z1, the acoustic impedance of the micro-channel 16 filled with the heat transfer material is Zm, the acoustic impedance of the second acoustic matching layer 15 is Z2, the acoustic impedance of the third acoustic matching layer 17 is Z3, and the acoustic impedance of the backing material 18 is Z4, it is desirable that the materials of the respective parts are selected such that Z1>Z2>Zm>Z3>Z4 is satisfied.

Here, given that the center wavelength of the ultrasonic waves to be transmitted and received is λ, it is desirable that the thickness of the vibrator is set to λ/2. Further, it is desirable that the thickness of the second acoustic matching layer 15 and the thickness of the third acoustic matching layer 17 are respectively set to λ/4. When the thickness of the micro-channel 16 is larger and the attenuation of ultrasonic waves in the heat transfer material within the micro-channel 16 is larger, the third acoustic matching layer 17 may be omitted. Further, the second acoustic matching layer 15 may be omitted depending on the acoustic impedance values of the respective parts.

Two circulation tubes 3a and 3b for circulation of the heat transfer material through the micro-channel 16, an electric cable 4 including plural coaxial cables and/or single wire cables, and a cable cover 5 for protecting them are connected to the casing 10. Here, the circulation tube 3a and an inflow hole formed in the third acoustic matching layer 17 and the backing material 18 forms a lead-in channel for leading in the heat transfer material, and the circulation tube 3b and an outflow hole formed in the third acoustic matching layer 17 and the backing material 18 forms a lead-out channel for leading out the heat transfer material.

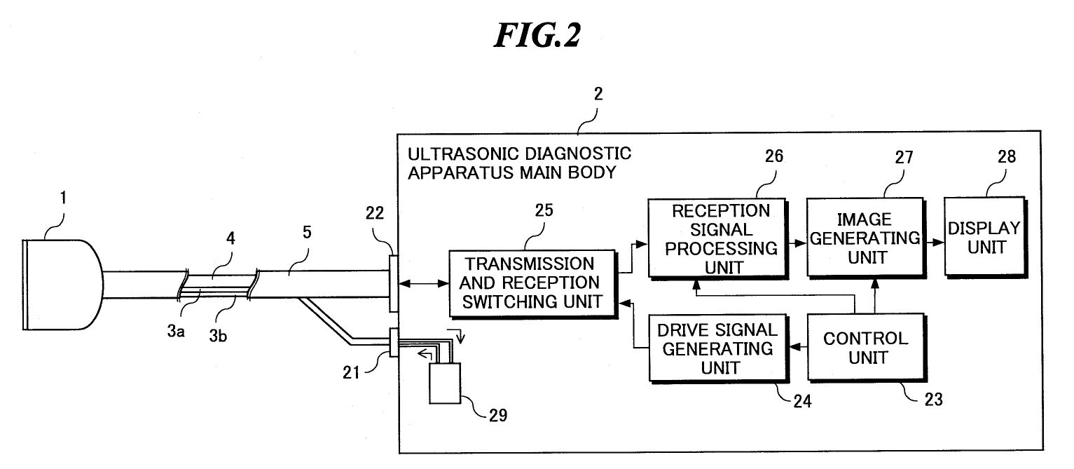

FIG. 2 shows a configuration of an ultrasonic diagnostic apparatus main body to which the ultrasonic probe according to any one of the first to third embodiments of the present invention is connected. As shown in FIG. 2, the circulation tubes 3a and 3b extending from the ultrasonic probe 1 are connected to an ultrasonic diagnostic apparatus main body 2 via a heat transfer material connector 21. In the ultrasonic diagnostic apparatus main body 2, a cooler 29 with a circulation pump cools the heat transfer material and supplies the cooled heat transfer material to the micro-channel 16 (FIG. 1) via the circulation tube 3a for heat transfer medium circulation, and collects the heat transfer material that has passed through the micro-channel 16 via the circulation tube 3b for heat transfer medium circulation. Thereby, the heat transfer material circulates between the ultrasonic probe 1 and the ultrasonic diagnostic apparatus main body 2.

Further, the ultrasonic probe 1 is electrically connected to the ultrasonic diagnostic apparatus main body 2 via the electric cable 4 and an electric connector 22. The electric cable 4 transmits drive signals generated in the ultrasonic diagnostic apparatus main body 2 to the respective ultrasonic transducers and transmits reception signals outputted from the respective ultrasonic transducers to the ultrasonic diagnostic apparatus main body 2.

The ultrasonic diagnostic apparatus main body 2 includes a control unit 23 for controlling the operation of the entire system including the ultrasonic probe 1 and the ultrasonic diagnostic apparatus main body 2, a drive signal generating unit 24, a transmission and reception switching unit 25, a reception signal processing unit 26, an image generating unit 27, a display unit 28, and the cooler 29 with the circulation pump. The drive signal generating unit 24 includes plural drive circuits (pulsers or the like), for example, and generates drive signals to be used for respectively driving the plural ultrasonic transducers. The transmission and reception switching unit 25 switches output of drive signals to the ultrasonic probe 1 and input of reception signals from the ultrasonic probe 1.

The reception signal processing unit 26 includes plural preamplifiers, plural A/D converters, and a digital signal processing circuit or CPU, for example, and performs predetermined signal processing of amplification, phasing addition, detection, etc. on the reception signals outputted from the respective ultrasonic transducers. The image generating unit 27 generates image data representing an ultrasonic image based on the reception signals on which the predetermined signal processing has been performed. The display unit 28 displays the ultrasonic image based on thus generated image data.

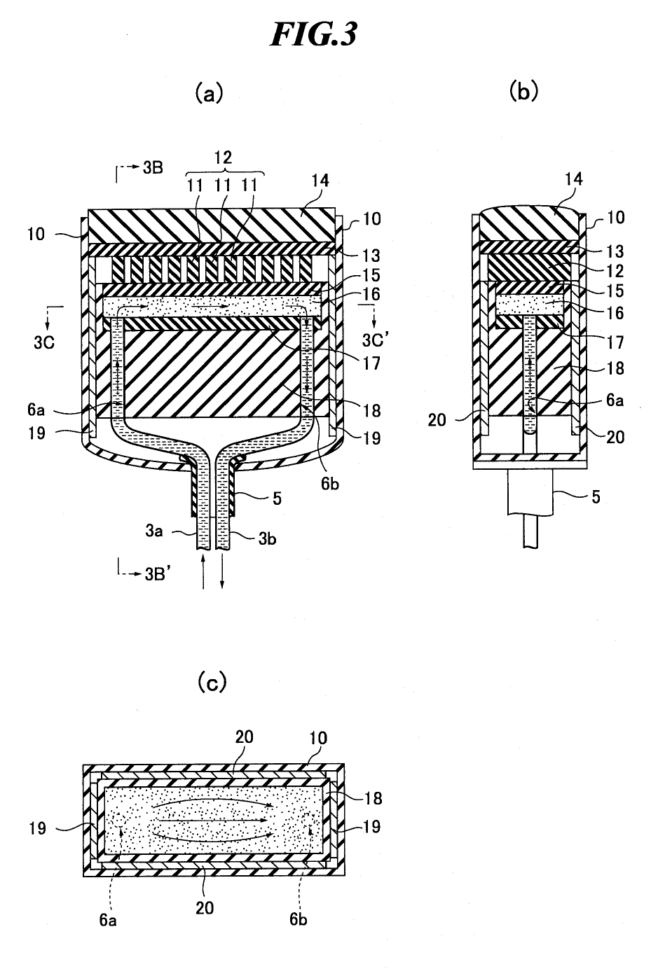

FIG. 3 (a) is a plan sectional view of the ultrasonic probe 1 according to the first embodiment of the present invention Further, FIG. 3 (b) is a side sectional view of the ultrasonic probe along the dashed-dotted line 3B-3B′ shown in FIG. 3 (a), and FIG. 3 (c) is a front sectional view along the dashed-dotted line 3C-3C′ shown in FIG. 3 (a). In FIG. 3 (a) to (c), the arrows indicate the flow directions of the heat transfer material.

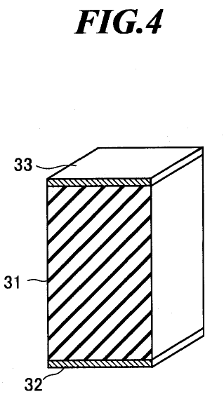

As shown in FIG. 3 (a), the ultrasonic transducer array 12 includes the plural ultrasonic transducers 11 arranged in a one-dimensional form. As shown in FIG. 4, each ultrasonic transducer includes a piezoelectric material 31 such as PZT (Pb(lead)zirconate titanate) and electrodes 32 and 33 formed on two opposite surfaces of the piezoelectric material. One of the electrodes 32 and 33 may be commonly connected among the plural ultrasonic transducers as a common electrode.

Referring to FIG. 3 again, the plural ultrasonic transducers 11 generate ultrasonic waves based on the drive signals respectively supplied from the ultrasonic diagnostic apparatus main body. Further, the plural ultrasonic transducers 11 receive ultrasonic echoes propagating from the object and generate electric signals. The electric signals are outputted to the ultrasonic diagnostic apparatus main body and processed as reception signals of the ultrasonic echoes. In order to reduce the interference among the plural ultrasonic transducers 11 and suppress the lateral vibration of the ultrasonic transducers 11 to allow the ultrasonic transducers 11 to vibrate only in the longitudinal direction, the spaces between the plural ultrasonic transducers 11 may be filled with a filling material.

At least one wiring pattern connected to the common electrode of the plural ultrasonic transducers 11 is formed on the two FPCs 19. One end of the wiring pattern is connected to the common electrode of the plural ultrasonic transducers 11 and the other end of the wiring pattern is connected to the ground lines of the plural coaxial cables. Further, plural wiring patterns respectively connected to the signal electrodes of the plural ultrasonic transducers 11 are formed on the two FPCs 20. One ends of the wiring patterns are respectively connected to the signal electrodes of the plural ultrasonic transducers 11 and the other ends of the wiring patterns are respectively connected to the hot lines of the coaxial cables. In FIG. 3, the cable for transmission of electric signals is omitted for easy understanding of the flow of the heat transfer material.

The first acoustic matching layer 13 formed on the front of the ultrasonic transducers 11 is formed of Pyrex (registered trademark) glass or an epoxy resin including metal powder, which easily propagates ultrasonic waves, for example, and provides matching of acoustic impedances between the object as a living body and the ultrasonic transducers 11. Thereby, the ultrasonic waves transmitted from the ultrasonic transducers 11 efficiently propagate within the object. Although the single-layer acoustic matching layer has been shown on the front of the ultrasonic transducers 11 in FIGS. 1 and 3, plural acoustic matching layers may be provided according to need.

The acoustic lens 14 is formed of silicone rubber, for example, and focuses an ultrasonic beam transmitted from the ultrasonic transducer array 12 and propagating through the acoustic matching layer 13, at a predetermined depth within the object.

The second acoustic matching layer 15 and the third acoustic matching layer 17 are also formed of Pyrex (registered trademark) glass or an epoxy resin including metal powder, and their acoustic impedances satisfy the above explained condition.

The backing material 18 is formed of a material having large acoustic attenuation such as an epoxy resin including ferrite powder, metal powder, or PZT powder, or rubber including ferrite powder, and promotes attenuation of unwanted ultrasonic waves generated from the ultrasonic transducers 11.

The micro-channel 16 is formed of a porous material such as porous ceramics. In FIG. 3, the micro-channel 16 is formed between the second acoustic matching layer 15 and the third acoustic matching layer 17, and both side surfaces and both end surfaces of the micro-channel 16 are covered by the backing material 18 for preventing outflow of the heat transfer material. Alternatively, a coating may be formed by employing a resin material or the like to cover both side surfaces and both end surfaces of the micro-channel 16 and further cover the upper surfaces and/or lower surfaces of the micro-channel 16 in the drawing. As the resin material, epoxy resin, urethane resin, silicone resin, polyimide resin, acrylic resin, or the like may be used.

The heat transfer material is a liquid for passing through the micro-channel 16 to absorb the heat generated from the ultrasonic transducers 11. As the heat transfer material, a material having good heat transference is used. For example, liquid paraffin, silicone oil, water, alcohol, mixture of water and alcohol, and fluorinated inert liquid may be used. Among them, liquid paraffin, silicone oil, and a fluorinated inert liquid (e.g., FLUORINERT (registered trademark) manufactured by Sumitomo 3M) are preferable, and in the embodiment, the liquid paraffin is used.

The inflow hole 6a and the outflow hole 6b for the heat transfer material are formed in the third acoustic matching layer 17 and the backing material 18. Further, the circulation tube 3a is connected to the inflow hole 6a and the circulation tube 3b is connected to the outflow hole 6b in the lower surface of the backing material 18. The heat transfer material introduced from the ultrasonic diagnostic apparatus main body via the circulation tube 3a into the ultrasonic probe sequentially passes the inflow hole 6a, the micro-channel 16, and the outflow hole 6b, and is collected in the ultrasonic diagnostic apparatus main body via the circulation tube 3b.

As described above, in the embodiment, the heat transfer material cooled in the ultrasonic diagnostic apparatus main body 2 is flown through the micro-channel 16 of the ultrasonic probe 1. Although the micro-channel 16 contacts the plural ultrasonic transducers 11 via the second acoustic matching layer 15, the thickness of the second acoustic matching layer 15 is smaller than (about one-half of) the thickness of the ultrasonic transducers (vibrators) 11, and thus, the heat generated by the ultrasonic transducers 11 is efficiently absorbed by the heat transfer material.

Therefore, the plural ultrasonic transducers 11 can be uniformly cooled, and the central part of the ultrasonic transducer array 12, in which heat especially tends to stay, can be sufficiently and evenly cooled. Thereby, the temperature distribution in the plural ultrasonic transducers 11 is averaged and the influence by the temperature on the ultrasonic transmission and reception operation (sensitivity variations or the like) can be reduced.

Next, the second embodiment of the present invention will be explained.

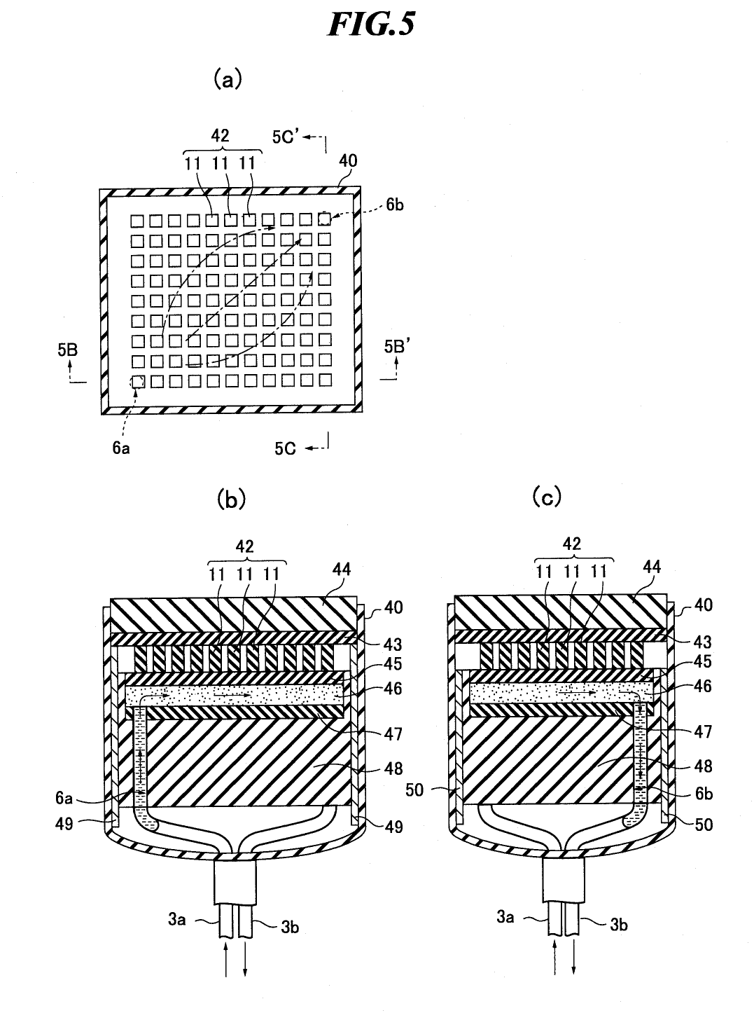

FIG. 5 (a) is a front view showing an interior of a head part of the ultrasonic probe according to the second embodiment of the present invention. Further, FIG. 5 (b) is a plan sectional view of the ultrasonic probe along the dashed-dotted line 5B-5B′ shown in FIG. 5 (a), and FIG. 5 (c) is a side sectional view of the ultrasonic probe along the dashed-dotted line 5C-5C′ shown in FIG. 5 (a). In FIG. 5 (a), an acoustic matching layer 43 and an acoustic lens 44 shown in FIG. 5 (b) are omitted.

As shown in FIG. 5 (a), the ultrasonic probe according to the second embodiment of the present invention has an ultrasonic transducer array 42 in which plural ultrasonic transducers 11 are two-dimensionally arranged, and accordingly, the micro-channel configuration formed within the ultrasonic probe is different from that in the first embodiment. The connection configuration between the ultrasonic probe and the ultrasonic diagnostic apparatus main body are the same as that have been explained with reference to FIG. 2.

The head part of the ultrasonic probe according to the second embodiment of the present invention includes a casing 40, the ultrasonic transducer array 42 including plural ultrasonic transducers 11, a first acoustic matching layer 43, an acoustic lens 44, a second acoustic matching layer 45, a micro-channel 46 for flowing a liquid heat transfer material (heat transfer medium), a third acoustic matching layer 47, a backing material 48, flexible printed circuits (FPCs) 49 connected to a common electrode of the plural ultrasonic transducers 11, and FPCs 50 connected to signal electrodes of the plural ultrasonic transducers 11. Further, the ultrasonic probe is connected to the ultrasonic diagnostic apparatus main body via circulation tubes 3a and 3b and an electric cable. The materials forming the first acoustic matching layer 43, the acoustic lens 44, the second acoustic matching layer 45, the third acoustic matching layer 47, and the backing material 48 and functions thereof are the same as those in the first embodiment.

In the ultrasonic transducer array 42, plural ultrasonic transducers 11 are arranged in a two-dimensional matrix form. As shown in FIG. 4, each ultrasonic transducer 11 includes a piezoelectric material 31 and electrodes 31 and 32 formed both sides of the piezoelectric material 31. One of the electrodes 31 and 32 may be commonly connected among the plural ultrasonic transducers as a common electrode. In order to reduce the interference among the plural ultrasonic transducers 11 and suppress the lateral vibration of the ultrasonic transducers 11 to allow the ultrasonic transducers 11 to vibrate only in the longitudinal direction, the spaces between the plural ultrasonic transducers 11 may be filled with a filling material.

At least one wiring pattern connected to the common electrode of the plural ultrasonic transducers 11 are formed on the two FPCs 49. One end of the wiring pattern is connected to the common electrode of the plural ultrasonic transducers 11 and the other end of the wiring pattern is connected to the ground lines of the plural coaxial cables. Further, plural wiring patterns respectively connected to the signal electrodes of the plural ultrasonic transducers 11 are formed on the two FPCs 50. One ends of the wiring patterns are respectively connected to the signal electrodes of the plural ultrasonic transducers 11 and the other ends of the wiring patterns are respectively connected to the hot lines of the coaxial cables. In FIG. 5, the cable for transmission of electric signals is omitted for easy understanding of the flow of the heat transfer material.

The micro-channel 46 is formed of a porous material such as porous ceramics. In FIG. 5, the micro-channel 46 is formed between the second acoustic matching layer 45 and the third acoustic matching layer 47, and four side surfaces of the micro-channel 46 are covered by the backing material 48 for preventing outflow of the heat transfer material. Alternatively, a coating may be formed by employing a resin material or the like to cover four side surfaces of the micro-channel 46 and further cover the upper surfaces and/or lower surfaces of the micro-channel 46 in the drawing. As the resin material, epoxy resin, urethane resin, silicone resin, polyimide resin, acrylic resin, or the like may be used.

In the embodiment, FLUORINERT is used as the heat transfer material. The inflow hole 6a and the outflow hole 6b for the heat transfer material are formed in the third acoustic matching layer 47 and the backing material 48. Further, the circulation tube 3a is connected to the inflow hole 6a and the circulation tube 3b is connected to the outflow hole 6b in the lower surface of the backing material 48. The heat transfer material introduced from the ultrasonic diagnostic apparatus main body via the circulation tube 3a into the ultrasonic probe is led into the micro-channel 46 through the inflow hole 6a, and two-dimensionally spreads as shown in FIG. 5 (a). Then, the heat transfer material flows into the outflow hole 6b formed in a position diagonally opposing the inflow hole 6a on the front of the ultrasonic probe, and is collected in the ultrasonic diagnostic apparatus main body via the circulation tube 3b.

In the two-dimensional ultrasonic transducer array as shown in FIG. 5 (a), the heat generated from the ultrasonic transducers located inner side is especially hard to disperse, and the heat especially tends to stay around the center. However, according to the embodiment, the heat transfer material is flown through the micro-channel 46 in contact with the plural ultrasonic transducers 11 via the second acoustic matching layer 45 having a relatively small thickness, and thereby, even the ultrasonic transducers around the center where the heat tends to stay can be sufficiently cooled. Therefore, the production of a temperature gradient can be suppressed in the two-dimensional ultrasonic transducer array, and thus, the influence due to temperature (e.g., sensitivity variations or the like) can be reduced.

In the embodiment, the inflow hole 6a and the outflow hole 6b are formed in two locations at corners of the third acoustic matching layer 47 and the backing material 48, however, the inflow hole and the outflow hole may be formed in other locations as long as the heat transfer material can be smoothly circulated. Further, two or more inflow holes and/or two or more outflow holes may be provided.

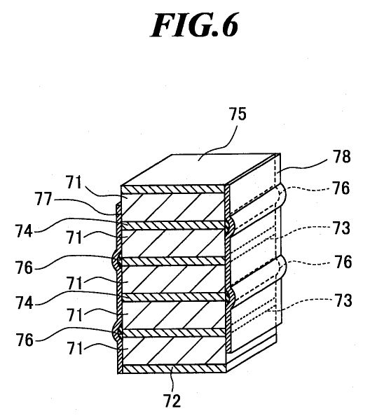

Next, the third embodiment of the present invention will be explained. In the third embodiment, an ultrasonic transducer including a multilayered piezoelectric material shown in FIG. 6 is used in place of the ultrasonic transducer including the single-layer piezoelectric material shown in FIG. 4 in the ultrasonic probe shown in FIG. 3 or FIG. 5.

The multilayered ultrasonic transducer shown in FIG. 6 includes plural piezoelectric material layers 71 formed of PZT or the like, a lower electrode layer 72, internal electrode layers 73 and 74, an upper electrode layer 75, insulating films 76, and side electrodes 77 and 78.

The lower electrode layer 72 is connected to the side electrode 77 on the left side in the drawing and insulated from the side electrode 78 on the right side in the drawing. Further, the internal electrode layers 73 and 74 are alternately inserted between the plural piezoelectric material layers 71. The internal electrode layers 73 are connected to the side electrode 78 and insulated from the side electrode 77 by the insulating films 76. On the other hand, the internal electrode layers 74 are connected to the side electrode 77 and insulated from the side electrode 78 by the insulating films 76. Furthermore, the upper electrode layer 75 is connected to the side electrode 78 and insulated from the side electrode 77. The plural electrodes of the ultrasonic transducer are thus formed, and thereby, five sets of electrodes for applying electric fields to the five layers of piezoelectric material layers 71 are connected in parallel. The number of the piezoelectric material layers is not limited to five as shown in FIG. 6, but two to four or six or more layers may be provided.

In the multilayered ultrasonic transducer (here, also referred to as “element”), areas of facing electrodes are larger than those in the single-layer element, and the electric impedance becomes lower. Therefore, the multilayered element operates more efficiently for an applied voltage than the single-layer element. Specifically, given that the number of the piezoelectric material layers is N (N=5 in FIG. 6), the number of the piezoelectric material layers is N times the number of the single-layer element and the thickness of each piezoelectric material layer is 1/N times the thickness thereof, and the electric impedance of the element is 1/N² times the electric impedance thereof. Therefore, the electric impedance of the element can be adjusted by increasing and decreasing the number of stacked layers of the piezoelectric material layers, and thus, the electric impedance matching with the drive circuit and/or the preamplifier can be easily provided and the sensitivity can be improved. On the other hand, the capacitance increases due to stacked form of the element, and the amount of heat generated from each element increases.

According to the embodiment, the heat transfer material is flown through the micro-channel 16 shown in FIG. 3 or the micro-channel 46 shown in FIG. 5 and the respective elements can be efficiently cooled, even when the amount of heat generated from the multilayered element increases. Therefore, the temperature rise of the ultrasonic probe can be suppressed.

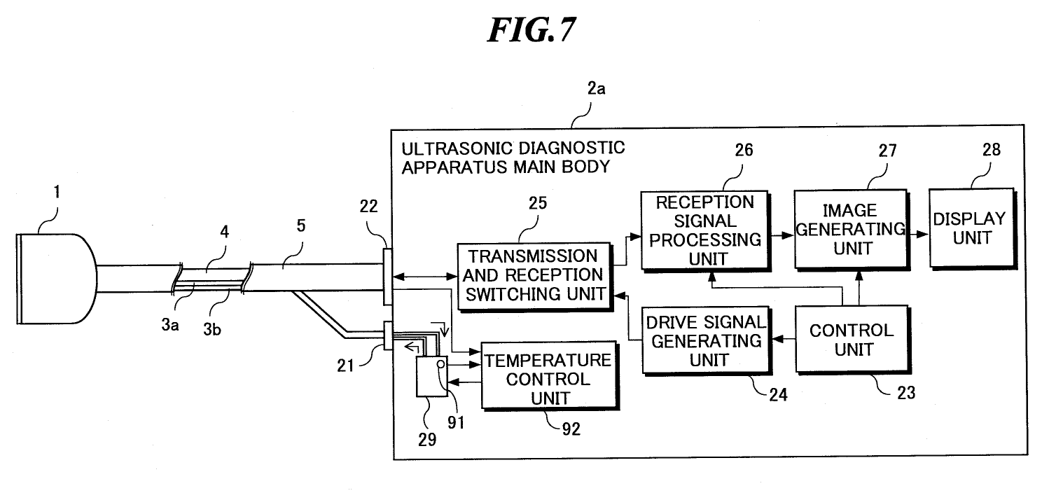

Next, a modified example of the ultrasonic diagnostic apparatus main body, to which the ultrasonic probe according to any one of the first to third embodiments of the present invention is connected, will be explained with reference to FIG. 7.

The ultrasonic diagnostic apparatus main body 2a shown in FIG. 7 further has a temperature sensor 91 and a temperature control unit 92 compared to the ultrasonic diagnostic apparatus main body 2 shown in FIG. 2. The rest of the configuration is the same as that shown in FIG. 2.

The temperature sensor 91 includes a thermistor, thermocouple, or the like. The temperature sensor 91 is attached to the cooler 29 with the circulation pump, and senses the temperature of the heat transfer material collected from the ultrasonic probe 1 via the circulation tube 3b. The temperature control unit 92 obtains a value on the temperature of the heat transfer material based on a signal outputted from the temperature sensor 91, and controls the operation of the cooler 29 with the circulation pump based on the obtained value. For example, when the obtained value on the temperature of the heat transfer material exceeds a predetermined value, the temperature control unit 92 lowers the preset temperature of the cooler or increases the pressure of the circulation pump for increasing the flow rate of the heat transfer material within the ultrasonic probe 1. Alternatively, the cooler 29 with the circulation pump may be operated only when the obtained value on the temperature of the heat transfer material exceeds the predetermined value.

According to the embodiment, since the operation of the cooler 29 with the circulation pump is feedback-controlled based on the temperature of the heat transfer material, the temperature of the heat transfer material can be easily kept in a certain range and the operation cost of the cooler 29 with the circulation pump can be reduced. As a modified example of the ultrasonic diagnostic apparatus main body shown in FIG. 7, a calculating unit for calculating the temperature based on the sensing result of the temperature sensor 91 is provided in place of the temperature control unit 92, and the control unit 23 may control the cooler 29 with the circulation pump based on a calculation result thereof.

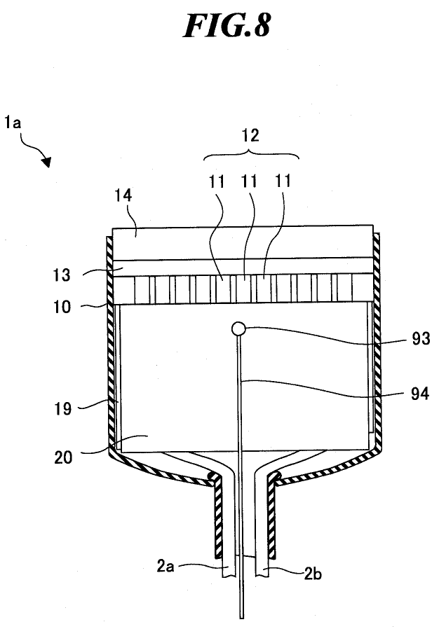

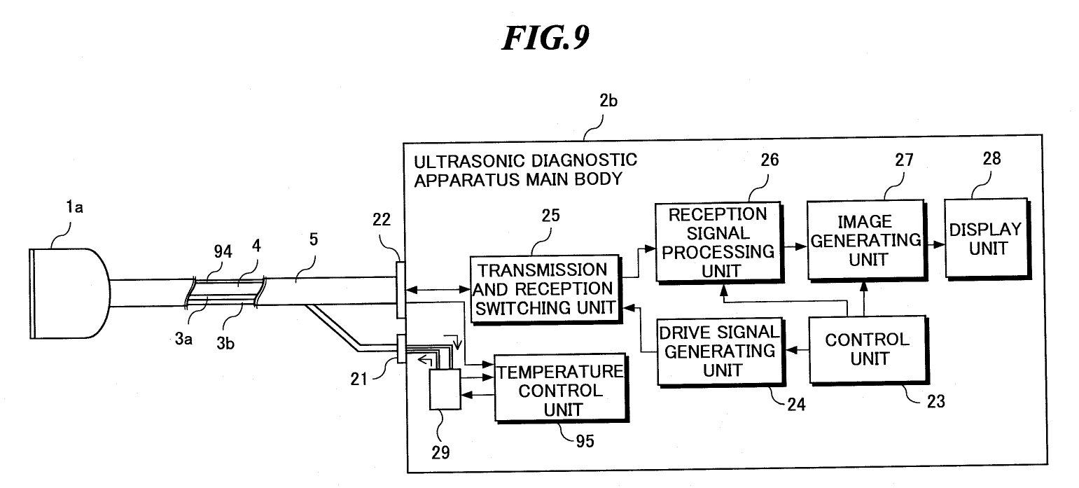

Next, an ultrasonic probe according to the fourth embodiment of the present invention will be explained with reference to FIGS. 8 and 9. FIG. 8 is a plan view showing an interior of the ultrasonic probe according to the fourth embodiment of the present invention, and FIG. 9 shows a configuration of an ultrasonic diagnostic apparatus main body to which the ultrasonic probe shown in FIG. 8 is connected.

As shown in FIG. 8, the ultrasonic probe 1a according to the embodiment further includes a temperature sensor 93 for sensing the temperature within the ultrasonic probe compared to the ultrasonic probe 1 shown in FIGS. 1 and 3. The rest of the configuration is the same as the ultrasonic probe 1 shown in FIGS. 1 and 3.

The temperature sensor 93 includes a thermistor, thermocouple, or the like, and is attached to the surface of the FPC 20. Alternatively, the temperature sensor 93 may be disposed in or on the backing material. In either case, the temperature sensor 93 is desirably located as close as possible to the micro-channel (16 in FIG. 3 or 46 in FIG. 5) or the ultrasonic transducer 11. The temperature sensor 93 is electrically connected to an ultrasonic diagnostic apparatus main body 2b (FIG. 9) by a lead wire 94.

As shown in FIG. 9, the ultrasonic diagnostic apparatus main body 2b to be used in the embodiment has a temperature control unit 95. The rest of the configuration of the ultrasonic diagnostic apparatus main body 2b is the same as that of the ultrasonic diagnostic apparatus main body 2 shown in FIG. 2.

The temperature control unit 95 obtains a value on the temperature of the heat transfer material based on a sensing result of the temperature sensor 93 received via the lead wire 94, and controls the operation of the cooler 29 with the circulation pump based on the obtained value such that the temperature of a head part 4 falls within a desired range. For example, when the obtained value on the temperature within the head part 4 exceeds a predetermined value, the temperature control unit 95 lowers the preset temperature of the cooler or increases the pressure of the circulation pump. Alternatively, the cooler 29 with the circulation pump may be operated only when the obtained value on the temperature within the head part 4 exceeds the predetermined value.

According to the embodiment, since the operation of the cooler 29 with the circulation pump is feedback-controlled based on the temperature within the head part of the ultrasonic probe 1a, the temperature within the head part can be controlled more accurately and the operation cost of the cooler 29 with the circulation pump can be reduced. Also in the embodiment, a calculating unit for calculating the temperature within the head part based on the sensing result of the temperature sensor 93 may be provided in place of the temperature control unit 95, and the control unit 23 may control the cooler 29 with the circulation pump based on a calculation result thereof.

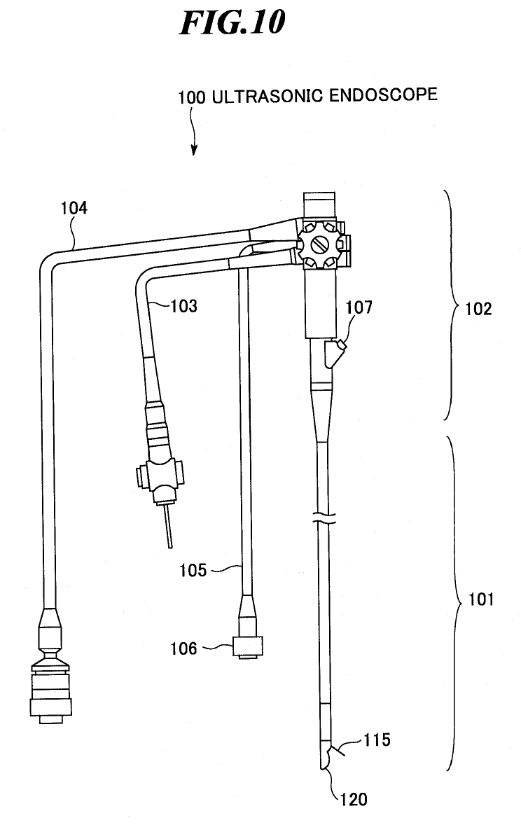

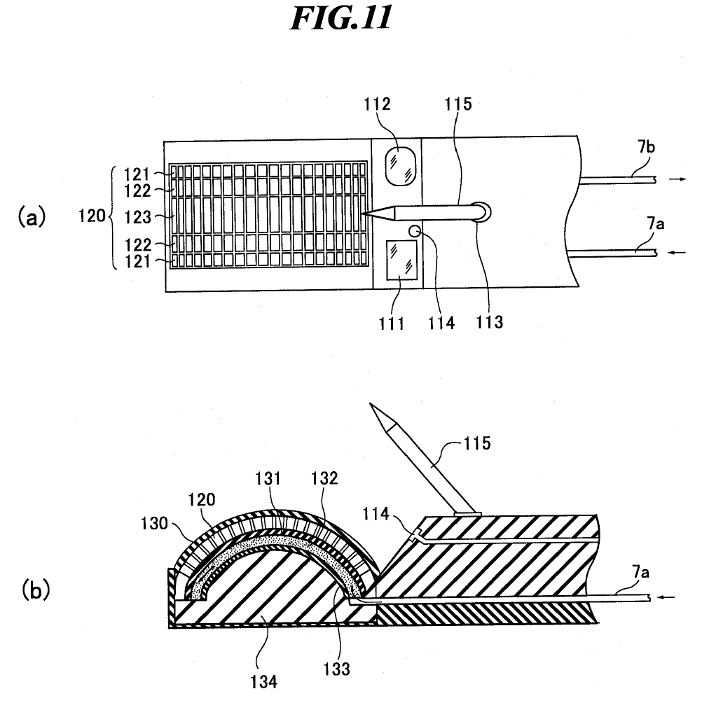

Next, an ultrasonic endoscope according to one embodiment of the present invention will be explained with reference to FIGS. 10 and 11. The ultrasonic endoscope is an instrument having an ultrasonic probe for intracavitary provided at the leading end of an insertion part of an endoscopic examination device for optical observation of the intracavitary of the object. The ultrasonic endoscope is connected to the ultrasonic diagnostic apparatus main body in the same way as the ultrasonic probe in FIG. 2, 7 or 9 to configure an ultrasonic diagnostic apparatus.

FIG. 10 is a schematic diagram showing an appearance of the ultrasonic endoscope. As shown in FIG. 10, the ultrasonic endoscope 100 includes an insertion part 101, an operation part 102, a connecting cord 103, a universal cord 104, a circulation medium cable 105, and a circulation medium connector 106. The insertion part 101 of the ultrasonic endoscope 100 is an elongated tube formed of a material having flexibility for insertion into the body of the object. The operation part 102 is provided at the base end of the insertion part 101, connected to the ultrasonic diagnostic apparatus main body via the connecting cord 103, and connected to a light source unit via the universal cord 104.

FIG. 11 is an enlarged schematic diagram showing the leading end of the insertion part 101 shown in FIG. 10. FIG. 11 (a) is a plan view showing the upper surface of the leading end of the insertion part 101, and FIG. 11 (b) is a side sectional view showing the side surface of the leading end of the insertion part 101. In FIG. 11 (a), the acoustic matching layer 130 shown in FIG. 11 (b) is omitted.

As shown in FIG. 11, at the leading end of the insertion part, an observation window 111, an illumination window 112, a treatment tool passage opening 113, a nozzle hole 114, and an ultrasonic transducer array 120 are provided. A punctuation needle 115 is provided in the treatment tool passage opening 113. In FIG. 11 (a), an objective lens is fit in the observation window 111, and an input end of an image guide or a solid-state image sensor such as a CCD camera is provided in the imaging position of the objective lens. These configure an observation optical system. Further, an illumination lens for outputting illumination light to be supplied from the light source unit via a light guide is fit in the illumination window 112. These configure an illumination optical system.

The treatment tool passage opening 113 is a hole for leading out a treatment tool or the like inserted from a treatment tool insertion opening 107 provided in the operation part 102 shown in FIG. 10. Various treatments are performed within a body cavity of the object by projecting the treatment tool such as the punctuation needle 115 or forceps from the hole and operating it with the operation part 102. The nozzle hole 114 is provided for injecting a liquid (water or the like) for cleaning the observation window 111 and the illumination window 112. The ultrasonic transducer array 120 is a convex-type multi row array and includes plural ultrasonic transducers 121-123 arranged in five rows on a curved surface.

As shown in FIG. 11 (b), an acoustic matching layer 130 is provided in front of the ultrasonic transducer array 120. An acoustic lens is provided on the acoustic matching layer 130 according to need. Further, on the back of the ultrasonic transducer array 120, a second acoustic matching layer 131, a micro-channel 132 as a cooling mechanism for cooling plural ultrasonic transducers, a third acoustic matching layer 133, and a backing material 134 are provided.

In the embodiment, in order to cool the plural ultrasonic transducers, the micro-channel 132 is formed between the second acoustic matching layer 131 and the third acoustic matching layer 132, and a heat transfer material flowing through the micro-channel 132 cools the ultrasonic transducer array 120. Here, the second acoustic matching layer 131 and the third acoustic matching layer 133 are provided for matching of acoustic impedances in a transfer path of ultrasonic waves from the ultrasonic transducer array 120 via the micro-channel 132 to the backing material 134. Thereby, the ultrasonic waves released to the back of the ultrasonic transducers can be sufficiently absorbed by the backing material 134.

Also in the embodiment, as is the case of the first embodiment, given that the center wavelength of the ultrasonic waves to be transmitted and received is λ, it is desirable that the thickness of the ultrasonic transducer (vibrator) is set to λ/2. Further, it is desirable that the thickness of the second acoustic matching layer 131 and the thickness of the third acoustic matching layer 133 are respectively set to λ/4. when the thickness of the micro-channel 132 is larger and the attenuation of ultrasonic waves in the heat transfer material within the micro-channel 132 is larger, the third acoustic matching layer 133 may be omitted. Further, the second acoustic matching layer 131 may be omitted depending on the acoustic impedance values of the respective parts.

The micro-channel 132 is formed of a porous material such as porous ceramics. In FIG. 11, the micro-channel 132 is formed between the second acoustic matching layer 131 and the third acoustic matching layer 133, and both side surfaces of the micro-channel 132 are covered by the backing material 134 for preventing outflow of the heat transfer material. Alternatively, a coating may be formed by employing a resin material or the like to cover both side surfaces of the micro-channel 132 and further cover the upper surfaces and/or lower surfaces of the micro-channel 132 in the drawing. As the resin material, epoxy resin, urethane resin, silicone resin, polyimide resin, acrylic resin, or the like may be used.

A circulation tube 7a for supplying the heat transfer material is connected to one end surface of the micro-channel 132 via an inflow hole formed on the backing material 134, and a circulation tube 7b for collecting the heat transfer material is connected to the other end surface of the micro-channel 132 via an outflow hole formed on the backing material 134. The circulation tubes 7a and 7b are accommodated in a heat transfer material cable 105 (see FIG. 10) and connected to a cooling unit provided inside or outside of the ultrasonic diagnostic apparatus main body. The heat transfer material circulates between the micro-channel 132 and the cooling unit via the circulation tubes 7a and 7b.

As described above, since the heat transfer material is flown through the micro-channel 132, the respective ultrasonic transducers 121-123 can be directly cooled. Thereby, the temperature rise of the ultrasonic endoscope is suppressed and the safety in ultrasonic endoscopic examination can be improved.

In FIG. 11, the convex-type multirow array is shown as the ultrasonic transducer array 120, however, a radial-type ultrasonic transducer array in which plural ultrasonic transducers are arranged on a cylindrical surface or an ultrasonic transducer array in which plural ultrasonic transducers are arranged on a spherical surface may be used. Further, also in the ultrasonic endoscopic shown in FIG. 11, the temperature sensor for sensing the temperature in the leading end of the insertion part 101 may be provided in the vicinity of the micro-channel 132 or the ultrasonic transducer so as to feedback-control the cooling unit of the heat transfer material based on the signal outputted from the temperature sensor.

1. An ultrasonic probe comprising:
an ultrasonic transducer array including plural ultrasonic transducers for transmitting and receiving ultrasonic waves;
an acoustic matching layer provided on a front of said ultrasonic transducer array;
a cooling mechanism directly or indirectly provided on a back of said ultrasonic transducer array and including a porous member;
a backing material provided on the back of said ultrasonic transducer array via at least said cooling mechanism; and
channels for circulation of a liquid heat transfer material in said cooling mechanism.

2. The ultrasonic probe according to claim 1, further comprising:
a second acoustic matching layer provided between said ultrasonic transducer array and said cooling mechanism.

3. The ultrasonic probe according to claim 1, further comprising:
a second acoustic matching layer provided between said cooling mechanism and said backing material.

4. The ultrasonic probe according to claim 1, wherein said cooling mechanism further includes a partition wall film formed on a side surface and/or an end surface of said porous member.

5. The ultrasonic probe according to claim 1, wherein each of said plural ultrasonic transducers includes a piezoelectric material and two electrodes respectively formed on opposite two surfaces of said piezoelectric material.

6. The ultrasonic probe according to claim 1, wherein each of said plural ultrasonic transducers includes plural piezoelectric material layers, plural internal electrode layers formed between said plural piezoelectric material layers, and two electrodes respectively formed on opposite two surfaces of said plural piezoelectric material layers.

7. The ultrasonic probe according to claim 1, wherein said heat transfer material includes one of liquid paraffin, silicone oil, water, alcohol, mixture of water and alcohol, and fluorinated inert liquid.

8. The ultrasonic probe according to claim 1, wherein said channels include:
a lead-in channel for leading said heat transfer material into said cooling mechanism; and
a lead-out channel for leading out said heat transfer material from said cooling mechanism.

9. An ultrasonic endoscope comprising:
an ultrasonic transducer array provided in an insertion part formed of a material having flexibility to be inserted into a body cavity of an object to be inspected and including plural ultrasonic transducers for transmitting and receiving ultrasonic waves;
an acoustic matching layer provided on a front of said ultrasonic transducer array;
a cooling mechanism directly or indirectly provided on a back of said ultrasonic transducer array and including a porous member;
a backing material provided on the back of said ultrasonic transducer array via at least said cooling mechanism; and
channels for circulation of a liquid heat transfer material in said cooling mechanism.

10. An ultrasonic diagnostic apparatus comprising:
an ultrasonic probe including an ultrasonic transducer array including plural ultrasonic transducers for transmitting and receiving ultrasonic waves, an acoustic matching layer provided on a front of said ultrasonic transducer array, a cooling mechanism directly or indirectly provided on a back of said ultrasonic transducer array and including a porous member, a backing material provided on the back of said ultrasonic transducer array via at least said cooling mechanism, and channels for circulation of a liquid heat transfer material in said cooling mechanism;
drive signal supply means for supplying drive signals to said plural ultrasonic transducers, respectively;
signal processing means for generating image data representing an ultrasonic image by processing reception signals outputted from said plural ultrasonic transducers, respectively; and
heat transfer material circulating means connected to the channels of said ultrasonic probe, for collecting the heat transfer material from said ultrasonic probe, cooling the collected heat transfer material, and supplying the cooled heat transfer material to said ultrasonic probe.

11. The ultrasonic diagnostic apparatus according to claim 10, further comprising:
temperature sensing means for sensing a temperature of the heat transfer material collected by said heat transfer material circulating means; and
temperature control means for controlling an operation of said heat transfer material circulating means based on a sensing result of said temperature sensing means.

12. The ultrasonic diagnostic apparatus according to claim 10, further comprising:
temperature sensing means for sensing a temperature within said ultrasonic probe; and
temperature control means for controlling an operation of said heat transfer material circulating means based on a sensing result of said temperature sensing means.

13. An ultrasonic diagnostic apparatus comprising:
an ultrasonic endoscope including an ultrasonic transducer array provided in an insertion part formed of a material having flexibility to be inserted into a body cavity of an object to be inspected and including plural ultrasonic transducers for transmitting and receiving ultrasonic waves, an acoustic matching layer provided on a front of said ultrasonic transducer array, a cooling mechanism directly or indirectly provided on a back of said ultrasonic transducer array and including a porous member, a backing material provided on the back of said ultrasonic transducer array via at least said cooling mechanism, and channels for circulation of a liquid heat transfer material in said cooling mechanism;
drive signal supply means for supplying drive signals to said plural ultrasonic transducers, respectively;
signal processing means for generating image data representing an ultrasonic image by processing reception signals outputted from said plural ultrasonic transducers, respectively; and
heat transfer material circulating means connected to the channels of said ultrasonic endoscope, for collecting the heat transfer material from said ultrasonic endoscope, cooling the collected heat transfer material, and supplying the cooled heat transfer material to said ultrasonic endoscope.

14. The ultrasonic diagnostic apparatus according to claim 13, further comprising:
temperature sensing means for sensing a temperature of the heat transfer material collected by said heat transfer material circulating means; and
temperature control means for controlling an operation of said heat transfer material circulating means based on a sensing result of said temperature sensing means.

15. The ultrasonic diagnostic apparatus according to claim 13, further comprising:
temperature sensing means for sensing a temperature in the insertion part of said ultrasonic endoscope; and
temperature control means for controlling an operation of said heat transfer material circulating means based on a sensing result of said temperature sensing means.
